(19) United States
(12) Patent Application Publication
(10) Pub. No.: US 2005/0229272 A1
(43) Pub. Date: Oct. 13, 2005

(54) Compositions and methods for gene silencing

(76) Inventors: Monica Driscoll, Highland Park, NJ (US); Nektarios Tavernarakis, Heraklion (GR)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/169,287

(22) PCT Filed: Jan. 2, 2001
(86) PCT No.: PCT/US01/00126

§ 371 Date: Apr. 19, 2005

(30) Foreign Application Priority Data
Dec. 30, 1999 (US) 60173999

(51) Int. Cl. A01H 1/00; C12N 15/82; C07H 21/04; A61K 48/00

(52) U.S. Cl. 800/279; 514/44; 435/468; 435/455; 536/23.1

(57) ABSTRACT

DNA constructs are provided for disrupting gene expression in targeted organisms.

### FIELD OF THE INVENTION

This invention relates the fields of molecular biology and gene silencing. More specifically, the invention provides compositions and methods for heritable and inducible gene silencing in target organisms.

### BACKGROUND OF THE INVENTION

Several publications are referenced in this application in order to more fully describe the state of the art to which this invention pertains. Complete citations may be found at the end of the specification. The disclosure of each of these publications is incorporated by reference herein.

RNA interference was discovered by Guo and Kemhues in the course of attempts to use antisense RNA to block gene expression in the maternal germ line. To their surprise, they found that both antisense and sense RNA preparations induced remarkably precise phenocopies of the targeted gene. Since then, both the efficacy and apparent lack of strand specificity associated with this interference process have been borne out in many subsequent studies. The mystery surrounding the mechanism of interference was recently deepened with the discovery that double-stranded RNA (dsRNA) is at least an order of magnitude more potent at inducing interferences than are preparations of either single strand. The surprising properties of this interference mechanism prompted users to abandon the term “antisense” and to begin referring to the process merely as “RNA interference”. The robust nature of the interference effect and the high degree of specificity have allowed RNAi to gain wide acceptance as a reverse genetic tool.

To date, most of these studies entail the microinjection of dsRNA duplexes corresponding to particular segments of targeted genes. While these methods are effective most of the time, the phenotype is not inherited. Accordingly, subsequent generations of targeted organisms must be continuously microinjected in order to attain the desired gene silencing effects. Likewise the technique is limited to organisms which are amenable to injection or application of dsRNA. It is an object of the present invention to overcome these limitations of the prior art.

### SUMMARY OF THE INVENTION

The present invention is directed to materials and methods which facilitate gene silencing in targeted organisms. In one embodiment of the invention, a DNA construct encoding an inverted repeat gene is provided. The construct comprises i) a promoter element operably linked in a 5′ to 3′ direction to a first coding sequence and a second sequence, the first coding sequence being in a sense orientation, the second sequence being the first coding sequence in an antisense orientation linked to the 3′ end of the first coding sequence; and ii) a transcription termination element operably linked 3′ to said first and second coding sequences. In an alternative embodiment, the first coding sequence of the IR construct is in an antisense orientation and the second coding sequence is in a sense orientation. In a preferred aspect of the invention, the inverted repeat gene of the invention is inserted into an expression vector.

In a preferred embodiment, the DNA constructs of the invention contain an inducible promoter to maximize expression of the inverted repeat genes of the invention. It should be apparent to those of skill in the art however that any promoter which acts to drive expression of the inverted repeat genes are also within the scope of the invention. Promoters contemplated for use in the DNA construct described above include, without limitation, heat shock promoters, metallothioneine promoter, glucocorticoid promoter, CMV promoter, SV40 promoter, nervous system specific C. elegans promoters, such as unc-119, mec-4, odr-4, and muscle promoters such as unc-54, myo-2, act-1 and ben-1. Optionally, the DNA constructs of the invention may include a spacer sequence between the first coding and second sequences. Such spacer sequences can be about 300, 500, 700, 1000 or 1500 nucleotides in length.

In yet another aspect of the invention, host cells containing the DNA constructs encoding the inverted repeat genes of the invention are provided. Such cells include, by way of illustration, C. elegans, yeast, Dictostelium, drosophila, mice, plants, insects, human cells and other nematodes

Methods for production of phenocopy knock out mutants via introduction of an inverted repeat gene into a target organism are also provided. The inverted repeat genes of the invention may be introduced into target organisms, such as C. elegans, via a process selected from the group consisting of microinjection, soaking, and DNA coated particle bombardment. Suitable targets for gene silencing include the following: green fluorescent protein gene, C3782.5, F26F12.7, T14G8.1, efk-1, mec-4, unc-8, unc-119, degenerinis ZB770.1, T28B8.5, T28F24.2, C24G7.2 and T28D9.7.

Several exemplary IR gene construct expression vectors are provided herein. IR gene constructs for reducing or inhibiting the expression of the beta amyloid protein are shown in FIG. 4. IR gene construct expression vectors for inhibiting or reducing the expression of alpha-synuclein are provided in FIG. 5. An exemplary vector for inhibiting geminivirus infection in tomato is provided in FIG. 6.

According to one aspect of the present invention, a method is provided for inhibiting or preventing the production of a pre-determined protein in a living organism. The method comprises providing a vector encoding IR ds RNA molecules which are capable of binding specifically to an mRNA sequence of interest. The vectors encoding the IR gene constructs of the invention are administered to the living organism under conditions whereby the vector enters cells, is expressed and thereafter specifically binds to the nucleic acid encoding the protein of interest, in an amount sufficient to reduce or inhibit production of the protein of interest.

According to another aspect of the present invention, a method is provided for treating a pathological condition related to an abnormal accumulation of disease-associated proteins. Examples of such abnormal pathological conditions include, without limitation, Alzheimer's disease, Parkinson's and Huntington's Disease. The method comprises administering to a patient having such a pathological condition a pharmaceutical preparation comprising vector having an IR gene construct contained therein capable of entering a cell expressing the protein of interest. Expression of the IR gene construct results in the generation of a nucleic acid molecule which specifically binds to a nucleic acid encoding the protein of interest, in an amount sufficient to affect the level of production of the protein of interest, thereby alleviating the pathological condition.

According to another aspect of this invention, a pharmaceutical preparation is provided for treating a pathological condition related to the abnormal accumulation of disease-related proteins. This pharmaceutical preparation comprises, in a biologically compatible medium, a vector having an IR gene construct contained therein capable of entering a cell and causing targeted gene silencing, in an amount sufficient to inhibit or reduce the level of production of the disease-associated protein. The biologically compatible medium is preferably formulated to enhance the lipophilicity and membrane-permeability of the IR gene construct expression vector.

The use of an inverted repeat RNAi expression construct as a delivery vehicle exploits the ability of such a vector to continue to generate multiple dsRNA copies, thereby prolonging the expression of the inhibitory RNA molecules indefinitely in vivo. This feature presents a distinct advantage over that of conventional modes for introduction of dsRNA, which provide a nonrenewable source of dsRNA in a one time delivery system. The methods and IR RNAi expression vectors of the present invention provide notable advantages over currently available compounds and methods for treating diseases associated with the abnormal expression, or accumulation of proteins in cells observed in many viral and neurodegenerative disorders.

### BRIEF DESCRIPTION OF THE DRAWINGS

FIG. 1 provides a schematic drawing of an IR gene construct expression vector of the invention.

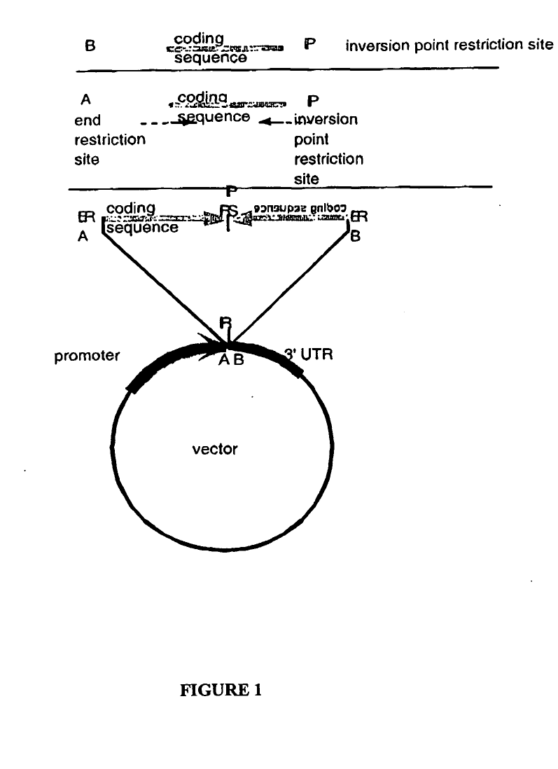

FIGS. 2A and 2B depict the strategy for generation of heritable and inducible RNAi in the nematode. A strong inducible promoter is fused to a direct inverted repeat gene. Upon induction of expression in transgenic animals harboring this gene, transcripts are generated which fold back in a uni-molecular reaction to generate double stranded RNA within all cells that express the heat shock gene. The size of the single stranded loop that occurs after foldback is not known (FIG. 2A). Construction of inducible inverted repeat genes is shown in FIG. 2B. Exon-rich genomic DNA (or CDNA) is amplified using two primers that introduce unique restriction sites at the fragment ends. One restriction site is used to generate the inverted repeat and is ultimately situated at the inversion point (IP). The other restriction site (designated as end) is ultimately used to join the inverted repeat to the vector. Amplified fragments are digested with the enzyme situated at the IP restriction site (IPRS) and ligated together. Digestion at the end restriction site (ERS) enables the fragment to be cloned into a similarly digested, CIAP-treated C. elegans expression vector. In our work, vector pPD49.78²², which includes the hsp16-2 promoter and the 3′ untranslated region of muscle myosin unc-54, was utilized (FIG. 2B).

FIGS. 3A and 3B show that double stranded RNA synthesized in vivo can disrupt C. elegans gene expression. FIG. 3A: Enzymatic assay for in vivo RNAi-induced disruption of the eEf2 kinase gene. CeEFK-1 activity was assayed as described¹¹ in reactions in which 0.5 μg rabbit reticulocyte eEF-2 was added to worm protein extracts. Arrow indicates the eEF-2 protein position. Lane 1, Wild type; lane 2, line harboring extrachromosomal hsp16-2ₚCefk-1 (IR), non-heat shocked; lane 3, a transgenic line harboring extrachromosomal parental vector pPD49.78, heat shocked; lane 4, line harboring extrachromosomal hsp16-2ₚCefk-1(IR), heat shocked; lane 5, Tc1 insertion Cefk-1 mutant. FIG. 3B: Use of in vivo RNAi to disrupt GFP expression in neurons and pharyngeal muscle. Progeny of transgenic lines harboring extrachromosomal unc-119ₚGFP (panels 1, 4; unc-119 is expressed in all neurons¹¹), integrated mec-4ₚGFP (panels 2,5; mec-4 is expressed in six touch sensory neurons¹⁴) or myo-2ₚGFP (panels 3, 6; myo-2 is expressed in pharyngeal muscle¹⁶) and hsp16-2ₚGFP(IR) were compared at 20° C. or consequent to parental heat shock at the L4 stage (35° C., 4 h). Progeny of similarly heat-shocked unc119ₚGFP, mec-4ₚGFP or myo-2ₚGFP lines exhibited no apparent reduction in intensity of neuronal fluorescence (data not shown). In parallel conventional RNAi experiments, 6 of 210 progeny of an unc-119ₚGFP parent, 11 of 270 progeny of a mec-4ₚGFP parent, and 57 of 240 progeny of a myo-2ₚGFP parent exhibited detectable reduction in GFP signal.

FIG. 4 is a schematic diagram of an IR gene construct expression vector suitable for inhibiting or reducing expression of the beta-amyloid protein.

FIG. 5 is a schematic diagram of an IR gene construct expression vector suitable for inhbiting or reducing expression of the a-synuclein protein.

FIG. 6 is an schematic diagram of an IR gene construct expression vector suitable for expressing the NP protein of a tomato geminivirus thereby inhibiting viral replication in the targeted tomato plant.

The detailed description set forth below describes preferred methods for practicing the present invention. Methods for selecting and preparing the IR gene constructs of the invention and expression vectors containing the same are described, as well as methods for administering the IR gene construct containing compositions in vivo. Specific in vitro and in vivo diagnostic and therapeutic applications of the IR gene construct compositions are also set forth.

### DETAILED DESCRIPTION OF THE INVENTION

Double-stranded RNA interference (RNAi) is an effective method for disrupting specific gene expression in C. elegans and other organisms¹⁻⁵. However, this powerful reverse genetics tool is most often employed in nematodes and plants because introduced dsRNA is not stably inherited¹. Another difficulty is that late-acting genes are not as efficiently knocked-out by RNAi as embryonically expressed genes. This may be due to a lowering of the concentration of dsRNA as cellular division proceeds during organismal development¹. In particular, some neuronally expressed genes appear refractory to dsRNA-mediated interference. It is an object of the invention to extend the applicability of RNAi by the controlled in vivo expression of heritable inverted-repeat (IR) genes. The efficacy of in vivo-driven RNAi has been assessed in three situations for which heritable, inducible RNAi would be advantageous: 1) production of large numbers of animals deficient for gene activities required for viability or reproduction, 2) generation of large populations of phenocopy mutants for biochemical analysis, and 3) effective gene inactivation in the nervous system. It is demonstrated herein that heritable inverted-repeat genes confer potent and specific gene inactivation for each of these applications, significantly broadening the already remarkable utility of RNAi for C. elegans reverse genetics.

Many neurodegenerative disorders result from the aberrant expression and/or accumulation of proteins to toxic levels. The IR gene constructs of the invention may be utilized to inhibit the expression of such proteins thereby alleviating the pathological symptoms of the disorder.

In a similar fashion, the IR gene construct expression vectors of the invention may be engineered to inhibit the expression of viral proteins in infected cells. Such viruses include both plant and animal viruses.

The IR gene constructs also have utility for the treatment of neoplastic diseases. For example, the aberrant expression of oncogenes in certain cancers may be targeted for gene silencing using the compositions and methods of the invention. Such oncogenes include, without limitation, ras, myc, myb, bcl-1, bcl-2, bcl-6, erb-a, erb-b, fgr, fos, src, lck, and lyn,

The IR gene construct expression vectors of the invention are also suitable for the generation of transgenic knock-out mice. Such mice provide ideal in vivo models for studying the contribution of particular genes to embryonic development, growth and disease.

Finally, the IR gene construct expression vectors of the invention may be used to advantage to generate disease resistant plants. For example, geminiviruses are plant pathogens that infect a wide range of vegetable crops in tropical and subtropical regions with devastating consequences (Brown et al. (1992) Plant Disease, 76:220-225). Traditional breeding methods have failed to generate cultivars that are resistant to geminiviruses. Transformation of target crops with the IR gene constructs of the invention encoding viral genes required for replication should effectively result in viral disease resistance.

I. Definitions

The following definitions are provided to aid in understanding the subject matter regarded as the invention.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

The terms “percent similarity”, “percent identity” and “percent homology” when referring to a particular sequence are used as set forth in the University of Wisconsin GCG software program.

The phrase “inverted repeat gene” as used herein refers to an expression construct containing a promoter element operably linked in the 5′ to 3′ direction to a first coding sequence in a sense orientation which is in turn operably linked to a second sequence consisting of the first coding sequence in an anti-sense orientation. Alternatively, the first coding sequence may be in an antisense orientation and the second coding sequence may be in a sense orientation. The first and second coding sequences range between 20 and 2500 nucleotides in length. Preferably the coding sequences may be between 100-300 nucleotides in length, 300-500 nucleotides in length, 500-800 nucleotides in length or 800-1500 nucleotides in length. Most preferably the first and second coding sequences are about 1000 nucleotides in length. The expression construct also contains 3′ regulatory regions which facilitate transcription of the inverted repeat gene in the targeted organism and the processing, expression, and translocation of its transcript. The IR gene constructs of the invention may optionally comprise a spacer sequence between the first and second coding sequences. Such spacer regions may be between 300-1000 nucleotides in length. In certain embodiments, the spacer comprises an intronic region.

The term “functional” as used herein implies that the nucleic or amino acid sequence is functional for the recited assay or purpose.

The phrase “consisting essentially of” when referring to a particular nucleotide or amino acid means a sequence having the properties of a given SEQ ID No:. For example, when used in reference to an amino acid sequence, the phrase includes the sequence per se and molecular modifications that would not affect the basic and novel characteristics of the sequence.

A “replicon” is any genetic element, for example, a plasmid, cosmid, bacmid, phage or virus, that is capable of replication largely under its own control. A replicon may be either RNA or DNA and may be single or double stranded.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism. Promoters may be inducible or constitutive. Inducible promoters include without limitation, heat shock promoter, metallothienine promoter, glucocorticoid promoter. Constitutive promoters suitable for the practice of the present invention in worms include, without limitation, nervous system promoters, such as unc-119, mec-4, odr-4, muscle promoters, such as unc-54 and myo-2 and general promoters, such as act-1 and ben-1. In higher organisms, promoters such as CMV are suitable. Other mammalian promoters are known to those of skill in the art and include for example, SV40, Mt promoter, glucocorticoid promoters.

When plants are targeted for gene silencing, the term “DNA construct” refers to genetic sequence used to transform plants and generate progeny transgenic plants. These IR gene constructs may be administered to plants in a viral or plasmid expression vector. The biolistic process of transformation is preferred for practice of the present invention. Other methods of delivery such as Agrobacterium T-DNA mediated transformation and transformation using electroporation are also contemplated to be within the scope of the present invention.

In a preferred embodiment of the present invention, the associated plant promoter is a strong and non tissue- or developmental-specific plant promoter (e.g. a promoter that strongly expresses in many or all tissue types). Examples of such strong, “constitutive” promoters include, but are not limited to, the CaMV 35S promoter, the T-DNA mannopine synthetase promoter, and their various derivatives.

In another embodiment of the present invention, it may be advantageous to engineer a plant with a gene construct operably associating a tissue- or developmental-specific promoter with a sequence encoding the desired enzyme. For example, where expression in photosynthetic tissues and organs are desired, promoters such as those of the ribulose bisphosphate carboxylase (RUBISCO) genes or chlorophyll a/b binding protein (CAB) genes may be used; where expression in seed is desired, promoters such as those of the various seed storage protein genes may be used; where expression in nitrogen fixing nodules is desired, promoters such those of the legehemoglobin or nodulin genes may be used; where root specific expression is desired, promoters such as those encoding for root-specific glutamine synthetase genes may be used (see Tingey et al., 1987, EMBO J.6:1-9; Edwards et al., 1990, Proc. Nat. Acad. Sci. USA 87:3459-3463).

In an additional embodiment of the present invention, it may be advantageous to transform a plant with an IR gene construct expression vector operably associating an inducible promoter with a sequence encoding the IR gene construct. Examples of such promoters are many and varied. They include, but are not limited to, those of the heat shock genes, the defense responsive gene (e.g., phenylalanine ammonia lyase genes), wound induced genes (e.g., hydroxyproline rich cell wall protein genes), chemically-inducible genes (e.g., nitrate reductase genes, gluconase genes, chitinase genes, etc.), dark-inducible genes (e.g., asparagine synthetase gene (Coruzzi and Tsai, U.S. Pat. No. 5,256,558, Oct. 26, 1993, Gene Encoding Plant Asparagine Synthetase) to name just a few.

The plant IR gene construct expression vectors of the invention may also comprise 3′ terminator sequences to stabilize the mRNA encoded by the construct. Such sequences include, without limitation, poly A sequences, and the os or nos 3′ terminator sequence.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specfically. The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield an primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

All amino-acid residue sequences represented herein conform to the conventional left-to-right amino-terminus to carboxy-terminus orientation.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. In other manners, the introduced nucleic acid may exist in the recipient cell or host organism only transiently.

A “clone” or “clonal cell population” is a population of cells derived from a single cell or common ancestor by mitosis.

A “cell line” is a clone of a primary cell or cell population that is capable of stable growth in vitro for many generations.

A “disease related protein may be a viral, bacterial or aberrant endogenously produced protein associated with a particular disease phenotype. Such human proteins include, without limitation, those set forth in Table I below:

TABLE I
Increased dosage/Gain-of-function disease genes in humans

```
  1. >gi|6681203|ref|NP_031894.1|| dystrophin, muscular dystrophy [Mus musculus]
  2. >gi|6995996|ref|NP_000483.2|| cystic fibrosis TM conductance regulator
  3. >gi|4753163|ref|NP_002102.2|| huntingtin [Homo sapiens]
  4. >gi|4502167|ref|NP_000475.1|| amyloid beta (A4) precursor protein
  5. >gi|2135246|pir||A56993 presenilin 2 - human
  6. >gi|4506435|ref|NP_000312.1|| retinoblastoma 1 (osteosarcoma) [Homo sapiens]
  7. >gi|1082578|pir||S50830 Machado-Joseph disease MJD1a protein - human
  8. >gi|1709040|sp|P54252|MJD1_HUMAN MACHADO-JOSEPH DISEASE PROTEIN 1
  9. >gi|3063388|dbj|BAA25751.1| Parkin [Homo sapiens]
10. >gi|4506113|ref|NP_000302.1|| prion protein (p27-30)[Homo sapiens]
11. >gi|2498924|sp|Q16637|SMN1_HUMAN SURVIVAL MOTOR NEURON PROTEIN 1
12. >gi|4507891|ref|NP_000542.1|| von Hippel-Lindau syndrome tumor suppressor
13. >gi|4507091|ref|NP_000335.1|| survival of motor neuron 1, telomeric
14. >gi|6166210|sp|Q92902|HPS_HUMAN HERMANSKY-PUDLAK SYNDROME PROTEIN
15. >gi|2228793|gb|AAC51731.1| Jagged1 [Homo sapiens]
16. >gi|904119|gb|AAB46416.1| S182 gene product, Alzheimer Disease Chr. 14
17. >gi|950348|gb|AAC42012.1|E5-1 protein Alzheimer Disease Chr. 1
18. >gi|4557365|ref|NP_000048.1|| Bloom syndrome protein [Homo sapiens]
19. >gi|4502839|ref|NP_000072.1|| Chediak-Higashi syndrome 1 [Homo sapiens]
20. >gi|6166193|sp|Q16595|FRDA_HUMAN FRATAXIN (FRIEDREICH'S ATAXIA PROTEIN)
21. >gi|6166210|sp|Q92902|HPS_HUMAN HERMANSKY-PUDLAK SYNDROME PROTEIN
22. >gi|4504455|ref|NP_000183.1|| Holt-Oram syndrome [Homo sapiens]
23. >gi|4557683|ref|NP_000207.1|| Kallmann syndrome 1 protein [Homo sapiens]
24. >gi|7531135|sp|Q12809|HERG_HUMAN VOLTAGE-GATED POTASSIUM CHANNEL
25. >gi|400664|sp|Q01968|OCRL_HUMAN LOWE'S OCULOCEREBRORENAL SYNDROME PROTEIN
26. >gi|1709040|sp|P54252|MJD1_HUMAN MACHADO-JOSEPH DISEASE PROTEIN 1
27. >gi|2135606|pir||I39294 McLeod syndrome-associated protein XK - human
28. >gi|4502321|ref|NP_000043.1|| ATPase, Cu++ transporting, (Menkes syndrome) [Homo sapiens]
29. >gi|5729770|ref|NP_000382.3|| ceroid-lipofuscinosis, neuronal 2, [Homo sapiens]
30. >gi|4557803|ref|NP_000262.1|| Niemann-Pick disease, type C1 [Homo sapiens]
31. >gi|4505339|ref|NP_002476.1|| Nijmegen breakage syndrome 1; nibrin [Homo
32. >gi|4505833|ref|NP_000287.1|| polycystic kidney disease 1 [Homo sapiens]
33. >gi|3126905|gb|AAC16004.1|polycystic kidney disease type II protein
34. >gi|4507091|ref|NP_000335.1|| survival of motor neuron 1, [Homo sapiens]
35. >gi|4506793|ref|NP_000323.1|| ataxin 1; spinocerebellar ataxia 1
36. >gi|4506795|ref|NP_002964.1|| ataxin 2; spinocerebellar ataxia 2
37. >gi|4506797|ref|NP_000324.1|| ataxin 7; spinocerebellar ataxia 7 [Homo sapiens]
38. >gi|4507891|ref|NP_000542.1|| von Hippel-Lindau syndrome tumor suppressor
39. >gi|482301|pir|A38080 Wilms tumor susceptibility protein WT1 - human
40. >gi|7513430|pir||A55197 Wiskott-Aldrich syndrome protein WASP - human
41. >gi|5174749|ref|NP_005996.1|| Wolfram syndrome [Homo sapiens]
42. >gi|6094278|sp|O60880|SH2A_DOMAIN PROTEIN 1A(DUNCAN'S DISEASE SH2-PROTEIN)
43. >gi|4502889|ref|NP_000077.1|| ceroid-lipofuscinosis, Spielmeyer-Vogt disease
44. >gi|189356|gb|AAA59964.1|Lowe Syndrome
45. >gi|515873 Membrane transport protein, McLeod Syndrome
46. >gi|34705|emb|CAA49145.1| Menkes Disease
47. >gi|307177|gb|AAA36206.1| protein kinase, Myotonic Dystrophy
48. >gi|292292|gb|AAA36212.1| Neurofibromatosis, Type 2
49. >gi|1163234|gb|AAA91041.1| Dpc4, Pancreatic Carcinoma
50. >gi|1314871|gb|AAC50481.1| retinitis pigmentosa GTPase regulator
51. >gi|1237181|gb|AAA98132.1| glypican, Simpson-Golabi-Behmel syndrome
52. >gi|624186|gb|AAA66242.1| survival motor neuron
53. >gi|1737213|gb|AAC52047.1| neuronal apoptosis inhibitory protein [Homo sapiens]
54. >gi|529662|emb|CAA55793.1| ataxin-1 [Homo sapiens] Spinocerebral Ataxia
55. >gi|731120|sp|P40337|VHL_HUMAN VON HIPPEL-LINDAU DISEASE TUMOR SUPPRESSOR (G7)
56. >gi|435421|gb|AAA03628.1| PAX-3 Waardenburg Syndrome
```

II. Preparation of Nucleic Acid Molecules Encoding the Inverted Repeat Genes of the Invention

Nucleic acid molecules comprising the inverted repeat genes of the invention may be prepared by two general methods: (1) synthesis from appropriate nucleotide triphosphates, or (2) isolation from biological sources. Both methods utilize protocols well known in the art. The availability of nucleotide sequence information for genes targeted for knock-out enables preparation of an isolated nucleic acid molecule of the invention by oligonucleotide synthesis. Synthetic oligonucleotides may be prepared by the phosphoramidite method employed in the Applied Biosystems 38A DNA Synthesizer or similar devices. The resultant construct may be purified according to methods known in the art, such as high performance liquid chromatography (HPLC). Long, double-stranded polynucleotides, such as a DNA molecule of the present invention, must be synthesized in stages, due to the size limitations inherent in current oligonucleotide synthetic methods. Thus, for example, a 1.9 kb double-stranded molecule may be synthesized as several smaller segments of appropriate complementarity. Complementary segments thus produced may be annealed such that each segment possesses appropriate cohesive termini for attachment of an adjacent segment. Adjacent segments may be ligated by annealing cohesive termini in the presence of DNA ligase to construct an entire 1.9 kb double-stranded molecule. A synthetic DNA molecule so constructed may then be cloned and amplified in an appropriate vector.

Nucleic acid sequences encoding the inverted repeat genes of the invention may be isolated from appropriate biological sources using methods known in the art. In one embodiment, a clone is amplified from a DNA expression library of from the desired species of origin. Suitable primers for this purpose are derived from sequences within the gene targeted for silencing. Such primers may be between 15 and 40 nucleotides in length. Alternative approaches for obtaining DNA for the inverted repeat genes of the invention, include cloning the inverted repeat fragments directly or chemically synthesizing the entire inverted repeat gene. In accordance with the present invention, nucleic acids having the appropriate level of sequence homology with the protein coding region of genes targeted for silencing may be identified by using hybridization and washing conditions of appropriate stringency. For example, hybridizations may be performed, according to the method of Sambrook et al., Molecular Cloning, Cold Spring Harbor Laboratory (1989), using a hybridization solution comprising: 5× SSC, 5× Denhardt's reagent, 1.0% SDS, 100 μg/ml denatured, fragmented salmon sperm DNA, 0.05% sodium pyrophosphate and up to 50% formamide. Hybridization is carried out at 37-42 C. for at least six hours. Following hybridization, filters are washed as follows: (1) 5 minutes at room temperature in 2× SSC and 1% SDS; (2) 15 minutes at room temperature in 2× SSC and 0.1% SDS; (3) 30 minutes-1 hour at 3-7 C. in 1× SSC and 1% SDS; (4) 2 hours at 42-65 C. in 1× SSC and 1% SDS, changing the solution every 30 minutes.

One common formula for calculating the stringency conditions required to achieve hybridization between nucleic acid molecules of a specified sequence homology (Sambrook et al., 1989) is as follows: Tₘ=81.5° C.+16.6Log [Na+]+0.41(% G+C)−0.63 (% formamide)−600/#bp in duplex. As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

Rather than direct cloning of genes targeted for silencing, an alternative approach entails identification of target genes by homology searches in available the available nucleic acid databases such as Genbank.

The inverted repeat genes of the present invention may be maintained as DNA in any convenient cloning vector. In a preferred embodiment, clones are maintained in a plasmid cloning/expression vector, such as pBluescript (Stratagene, La Jolla, Calif.), which is propagated in a suitable E. coli host cell. Other vectors suitable for the practice of the present invention, include, without limitation, pBlue/TOPO (Invitrogen), PCR-Blunt-TOPO (Invitrogen) and the pCDNA series from Invitrogen.

II. Selection and Preparation of Expression Vectors Containing the IR Gene Constructs of the Invention

Selection of a suitable sequence targeted for knock-out depends on knowledge of the nucleotide sequence of the target mRNA, or gene from which the mRNA is transcribed. Although targeting to mRNA is preferred and exemplified in the description below, it will be appreciated by those skilled in the art that other forms of nucleic acid, such as pre-mRNA or genomic DNA, may also be targeted.

Double-stranded IR transcripts should correspond to regions present in the transcript encoding the targeted protein. Such sequences include, but are not limited to 5′ untranslated regions, coding regions and the 3′ untranslated regions.

Various genetic regulatory control elements may be incorporated into the expression vectors containing the IR gene constructs of the invention to facilitate propagation in both eucaryotic and procaryotic cells. Different promoters may be utilized to drive expression of the IR gene construct sequences, the cytomegalovirus immediate early promoter being preferred for use in humans as it promotes a high level of expression of downstream sequences. Polyadenylation signal sequences are also utilized to promote mRNA stability. Sequences preferred for use in human cells include, but are not limited to, bovine growth hormone polyadenylation signal sequences or thymidine kinase polyadenylation signal sequences. Antibiotic resistance markers are also included in these vectors to enable selection of transformed cells. These may include, for example, genes that confer hygromycin, neomycin or ampicillin resistance.

A variety of different vectors are available for use in the methods of the invention. These include without limitation, those set forth in Table II. The listed vector have been utilized to express the indicated proteins. Conventional molecular biological techniques may be utilized to replace the protein coding sequence with the IR gene constructs of the invention.

TABLE II
VECTORS FOR USE IN THE METHODS OF
THE INVENTION

| Name | Description |
|---|---|
| pACCMVpLmP1(−)loxP-SSP | Adenoviral shuttle plasmid with |
| | unique restriction tag, SwaI, |
| | SfiI, Pmel, CMV promoter, pUC19 |
| | polylinker, mP1 splicing |
| | signal/poly A, loxP |
| pACCMVpLpA(−)loxP | Adenoviral shuttle plasmid, CMV |
| | promoter, pUC19 polylinker, |
| | SV40 splice/polyA, loxP |
| PACCMVpLPA(−)loxP-SSP | Adenoviral shuttle plasmid with |
| | unique restriction tag, Swal, |
| | Sfil, PmeI, CMV promoter, pUC19 |
| | polylinker, SV40 splice/polyA, |
| | loxP |
| pACpL + loxP | Adenoviral shuttle plasmid, no |
| | promoter |
| pACpL + loxP-SSP | Adenoviral shuttle plasmid, no |
| | promoter, SwaI, Sfil, Pmel |
| | unique site cluster |
| pAd-HSV | tkHSV tk, thymidine kinase, pAD |
| | BglII |
| pAdBgl II | Ampicillin Resistance, |
| | Recombinant Adenovirus |
| pAdEF1 alpha loxP | Adenovirus, EF 1 alpha |
| | promoter, loxP |
| pAdMCSlacZ | multiple cloning sites, lacZ |
| | gene |
| pAdMCSloxP | Adenoviral shuttle vector, |
| | polylinker, loxP |
| pAdMCSpA | multiple cloning sites, SV40 |
| | PolyA signal |
| pAdMCSpA/lacZ | multiple cloning sites, lacZ |
| | gene, SV40 poly(A) signal |
| pAdRSV4 | RSV promoter, multiple cloning |
| | sites, SV40 poly(A) |
| pNGVL1 | PstI, SalI, HindIII, EcoRV, |
| | BglII polylinker, kanamycin |
| | resistance |
| pNGVL1-CAT | chloramphenicol transferase |
| pNGVL1-hGM-CSF | human granulocyte-monocyte |
| | colony stimulating factor |
| pNGVL1-hpAP | human placental alkaline |
| | phospbatase |
| pNGVL1-mGM-CSF | mouse granulocyte-monocyte |
| | colony stimulating factor |
| pNGVL1-ntbeta-gal | nuclear targeted beta- |
| | galactosidase |
| pNGVL1-tk | thymidine kinase |
| pNGVL2 | polylinker deleted, kanamycin |
| | resistance |
| pNGVL3 | 12 site polylinker, kanamycin |
| | resistance |
| pNGVL3-4070a-env | Amphotropic 4070A virus env. |
| pNGVL3-gag-pol | retrovirus gag-pol helper |
| | plasmid, kanamycin resistant |
| pNGVL3-hFL | human full length Flt3 ligand |
| | cDNA |
| pNGVL3-hFLex | human Flt3 ligand, secreted |
| pNGVL3-hIL10 | human interleukin 10 |
| pNGVL3-hIL12 | human interleukin-12, internal |
| | ribosome entry site |
| pNGVL3-hIL15 | human interleukin-15 |
| pNGVL3-hIL2 | human interleukin-2 |
| pNGVL3-hIL2/IL15 | human IL2-IL15 fusion |
| pNGVL3-hIL4 | human interleukin 4 |
| pNGVL3-hIL7 | human interleukin 7 |
| pNGVL3-mFL | mouse FLT3 ligand |
| pNGVL3-mFLex | Extracellular domain of mouse |
| | FLT3 ligand |
| pNGVL3-mIL10 | mouse interleukin 10 |
| pNGVL3-mIL12 | mouse interleukin 12, internal |
| | ribosome entry site |
| pNGVL3-mIL15 | mouse interleukin-15 |
| pNGVL3-mIL2 | Mouse interleukin-2 |
| pNGVL3-mIL4 | mouse interleukin 4 |
| pNGVL3-mIL7 | mouse interleukin 7 |
| pNGVL3-shIL15R | soluble human interleukin 15 |
| | receptor |
| pNGVL3-smIL15R | soluble mouse interleukin-15 |
| | receptor |
| pNGVL4a | immunostimulatory sequence |
| pNGVL4b | immunostimulatory sequence |
| pNGVL5 | IL2 secretory signal peptide |
| pNGVL6a | IL2 secretion signal peptide |
| pNGVL7 | pNGVL7, CMV, tpa |
| RVNL3(+) | CMV promoter, ATG(−), non- |
| | episomal, retrovirus |
| | vector |

IV. Uses of Nucleic Acids Encoding Inverted Repeat Genes

Gene silencing provides a powerful technique for the elucidation of molecular and biochemical mechanisms associated with homeostasis, growth and development. The inverted repeat genes of the invention may be used to advantage to “knock out” the activity of any target gene, provided the sequence of the target gene is known. The inverted repeat gene constructs have been designed such that they are both inducible and heritable. Expression of the inverted repeat genes of the invention driven from strong inducible promoters facilitates the formation of a double stranded “snap back” RNA endogenously, thereby abrogating functional expression of the target gene.

Gene expression manipulation is extremely important to the pharmaceutical industry. Beneficial uses of this invention include specific gene inactivation for the investigation of gene function and reverse genetic studies. When C. elegans is used as the target organism, the compositions and methods of the invention facilitate the production of phenocopy mutants which can be induced also in offspring. Thus the invention provides for the production of large populations of nematodes that can be subjected to gene inactivation at any time during growth and development which in turn may be used to advantage for drug screening, large scale genetic mutant assessment, and biochemical studies. The invention also provides for disruption of gene activity, markedly improving current protocols which appear to be ineffective in neuronal “knock out”. The invention also provides for large scale preparations for analysis of the RNAi mechanism itself.

Additionally, this protocols described herein may be used to inactivate or disrupt gene activity in any organism. Any organism which may be transformed with exogenous DNA corresponding to a target gene having a defined promoter may be subjected to gene silencing using the compositions and methods of the present invention. Such organisms include, without limitation, yeast, Dictostelium, drosophila, mice, insects, plants, human cells and other nematodes. Such methods facilitate an analysis of specific gene function by phenocopy knockout. Additionally, other harmful or potentially harmful gene expression may be inhibited or prevented in accordance with the invention. Such genes include by way of illustration, genes required for oncogenisis, productive HIV infection and genes required for successful infection by a variety of pathogenic organisms.

The availability of sequence information encoding nucleic acids targeted for gene silencing enables the production of strains of laboratory mice carrying the IR gene constructs of the invention. Such mice provide an in vivo model for assessing growth development and disease. The compositions and methods provided herein enables the production of knockout mice in which the endogenous gene corresponding to the IR gene construct has been specifically inactivated. Methods of introducing IR gene construct expression vectors in laboratory mice are known to those of skill in the art. Three common methods include: 1. integration of retroviral vectors encoding the foreign gene of interest into an early embryo; 2. injection of DNA into the pronucleus of a newly fertilized egg; and 3. the incorporation of genetically manipulated embryonic stem cells into an early embryo. Production of the transgenic mice described above will faciliate the molecular elucidation of the role predetermined target genes play in embryonic development and disease.

A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring, in fact, possess some or all of that alteration or genetic information, then they, too, are transgenic animals.

The DNA used for altering expression of a target gene may be obtained by a wide variety of techniques that include, but are not limited to, isolation from genomic sources, preparation of cDNAs from isolated MRNA templates, direct synthesis, or a combination thereof. A type of target cell for transgene introduction is the embryonal stem cell (ES). ES cells may be obtained from pre-implantation embryos cultured in vitro (Evans et al., (1981) Nature 292:154-156; Bradley et al., (1984) Nature 309:255-258; Gossler et al., (1986) Proc. Natl. Acad. Sci. 83:9065-9069). Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

One approach to the problem of determining the contributions of individual genes and their expression products is to use IR gene constructs to selectively inactivate the wild-type gene in totipotent ES cells (such as those described above) and then generate transgenic mice. The use of gene-targeted ES cells in the generation of gene-targeted transgenic mice was described, and is reviewed elsewhere (Frohman et al., (1989) Cell 56:145-147; Bradley et al., (1992) Bio/Technology 10:534-539).

Techniques are available to inactivate or alter any genetic region to a mutation desired by using targeted homologous recombination to insert specific changes into chromosomal alleles. However, in comparison with homologous extrachromosomal recombination, which occurs at a frequency approaching 100%, homologous plasmid-chromosome recombination was originally reported to only be detected at frequencies between 10⁻⁶ and 10⁻³. Nonhomologous plasmid-chromosome interactions are more frequent occurring at levels 10⁵-fold to 10²-fold greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformant cells for homologous insertion, followed by screening of individual clones. Alternatively, a positive genetic selection approach has been developed in which a marker gene is constructed which will only be active if homologous insertion occurs, allowing these recombinants to be selected directly. One of the most powerful approaches developed for selecting homologous recombinants is the positive-negative selection (PNS) method developed for genes for which no direct selection of the alteration exists. The PNS method is more efficient for targeting genes which are not expressed at high levels because the marker gene has its own promoter. Non-homologous recombinants are selected against by using the Herpes Simplex virus thymidine kinase (HSV-TK) gene and selecting against its nonhomologous insertion with effective herpes drugs such as gancyclovir (GANC) or (1-(2-deoxy-2-fluoro-B-D arabinofluranosyl)-5-iodouracil, (FIAU). By this counter selection, the number of homologous recombinants in the surviving transformants can be increased.

Methods of use for the transgenic mice of the invention are also provided herein. Therapeutic agents for the treatment or prevention of disease may be screened in studies using transgenic mice harboring the IR gene constructs of the invention.

As mentioned previously, the IR gene construct expression vectors of the invention may be used for gene silencing in any organism which may be targeted with exogenous DNA. Uses of the expression vectors for the treatment of human and plant diseases is also exemplified herein.

V. Administration of Plasmid Vectors Producing the IR Genes of the Invention

The IR gene construct containing expression vectors as described herein are generally administered to a patient as a pharmaceutical preparation. The term “patient” as used herein refers to human or animal subjects.

The pharmaceutical preparation comprising the IR gene construct expression vector of the invention is conveniently formulated for administration with a acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of expression vector in the chosen medium will depend on the hydrophobic or hydrophilic nature of the medium, as well as the length and other properties of the vector molecule. Solubility limits may be easily determined by one skilled in the art.

As used herein, “biologically acceptable medium” includes any and all solvents, dispersion media and the like which may be appropriate for the desired route of administration of the pharmaceutical preparation, as exemplified in the preceding paragraph. The use of such media for pharmaceutically active substances is known in the art. Except insofar as any conventional media or agent is incompatible with the IR gene construct expression vectors to be administered, its use in the pharmaceutical preparation is contemplated.

Selection of a suitable pharmaceutical preparation depends upon the method of administration chosen. For example, IR gene construct expression vectors may be administered by direct injection into the region of the brain containing the targeted cell type. In this instance, a pharmaceutical preparation comprises the IR gene construct expression vector dispersed in a medium that is compatible with cerebrospinal fluid. In a preferred embodiment, artificial cerebrospinal fluid (148 mM NaCl, 2.9 mM KCl, 1.6 mM MgCl₂ 6H₂O, 1.7 mM CaCl₂, 2.2 mM dextrose) is utilized, and IR gene construct expression vectors are provided directly to neurons by intraventricular injection.

IR gene construct expression vectors for use in gene silencing may also be administered parenterally by intravenous injection into the blood stream, or by subcutaneous, intramuscular or intraperitoneal injection. Pharmaceutical preparations for parenteral injection are commonly known in the art. If parenteral injection is selected as a method for administering the IR gene construct expression vector, steps must be taken to ensure that sufficient amounts of the molecules reach their target cells to exert a biological effect. The lipophilicity of the IR gene construct expression vectors, or the pharmaceutical preparation in which they are delivered may have to be increased so that the molecules can cross the blood-brain barrier to arrive at their target locations. Furthermore, the IR gene construct expression vectors may have to be delivered in a cell-targeted carrier so that sufficient numbers of molecules will reach the target cells. Methods for increasing the lipophilicity of a molecule are known in the art, and include the addition of lipophilic groups to the IR gene construct expression vector.

Several techniques have been used to increase the stability, cellular uptake and biodistribution of DNA expression vectors. The expression vector of the present invention may be encapsulated in a lipophilic, targeted carrier, such as a liposome. One technique is to use as a carrier for the expression vector a liposomal preparation containing the cationic lipid N-[1-(2,3-dioleyloxy)propyl]-N,N,N-trimethyl ammonium chloride (D OT MA; lipofectin).

The vectors of the present invention may be complexed to liposomes. To further facilitate targeting of the IR gene construct expression vector, liposomes may be “studded” with antibodies specific for certain regions of the brain (Leserman et al., (1980) Nature 288:604). In a preferred embodiment, cationic liposomes are complexed with (1) the IR gene construct expression vector; and (2) antibodies specific for the desired region of the brain. Vector containing antibody-studded-liposome complexes are expected not only to be targeted and specifically expressed in the desired regions of the brain, but also to be expressed for indefinitely.

The pharmaceutical preparation is formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

The pharmaceutical preparation comprising the IR gene construct expression vector may be administered at appropriate intervals, for example, twice a day until the pathological symptoms are reduced or alleviated, after which the dosage may be reduced to a maintenance level. The appropriate interval in a particular case would normally depend on the condition of the patient.

When performing gene silencing in plants, the IR gene construct expression vector is delivered to plant cells using biolistic or Agrobacterium mediated DNA transfer. Selection and propagation of transformed plant cells is performed according to methods well known to those of ordinary skill in the art.

The following protocols are provided to facilitate the practice of the present invention.

Nematode strains. C. elegans strains were reared and maintained as described²¹. We constructed transgenic lines by injection of plasmid DNAs each at 100 ng/μl using standard protocols²². In all experiments we used plasmid pRF4²³, which harbors a dominant rol-6 allele that causes a readily distinguished roller phenotype in transgenic animals, as a co-transformation marker.

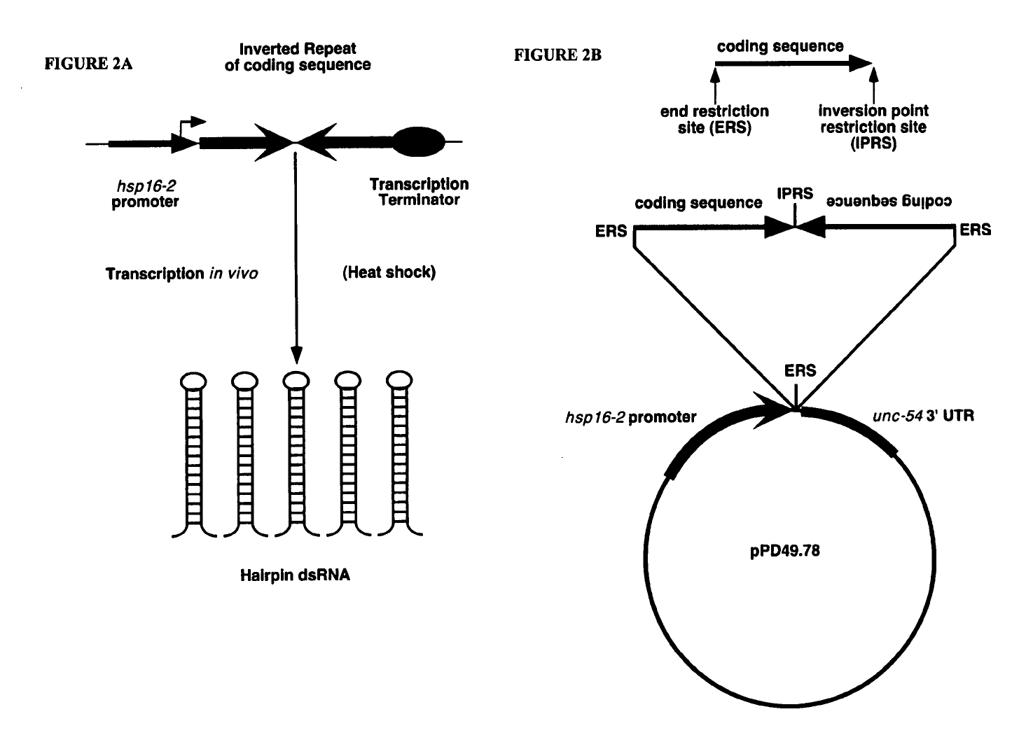

Construction of inverted repeat genes. We PCR amplified exon-rich genomic DNA (or cDNA) using two primers that introduce unique restriction sites at the fragment ends. We digested the amplified fragment with one of the enzymes and ligated to generate an inverted repeat (outlined in FIG. 2b). We then digested with the other enzyme, the restriction site for which is now positioned at the IR fragment ends, and ligated into CIAP-treated vector pPD49.78²², which includes the hsp16-2 promoter and the 3′ untranslated region of muscle myosin unc-54. The cDNA and genomic DNA we amplified for RNAi ranged from 0.58-1.45 kb in length. Alternative cloning strategies include: 1) digestion at two naturally occurring restriction sites to excise the gene fragment of interest with subsequent two-step ligation as above, or 2) direct tri-molecular ligation of the doubly digested fragment into CIAP-treated vector previously linearized with one of the enzymes at the fragment end. In an alternative embodiment spacer loops are included between the inverted repeat gene segments. We found the efficiency of cloning inverted repeats to be acceptable in the E. coli DH5 strain (in general, a few per hundred candidates screened) and relatively high in the E. coli SURE strain (Stratagene), a bacterial host tolerant of inverted repeats (about 1/20 candidate constructs correct). The hsp16-2ₚunc-8 (IR) construct, however, proved highly difficult to generate (1000 candidates screened, 0.58 kb of cDNA sequence in the repeat) for reasons that are not clear. Slower growing bacterial transformant colonies appear to have an enhanced chance of harboring the IR gene. The yield of plasmid DNA from IR genes harbored in E. coli DH5 strain is low (about 3-5 μg per 50 ml culture); when the SURE strain is the host, yields are improved (80-100 μg per 50 ml culture). While this method relates to PCR amplification of the target genes, it will be appreciated by those of skill in the art that other methods for obtaining the DNA for the inverted repeat genes of the invention are available. These include direct synthesis of the target gene on a DNA synthesizer and direct cloning using conventional hybridization and DNA isolation procedures.

RNA interference assays. For standard RNAi, we prepared dsRNA from cDNAs or coding sequence-rich genomic DNAs, 0.58-1.2 kb in length are injected into N2 adults/group as described¹. We scored progeny born to injected adults (10 adults per group) 12 hours or more after injection (older progeny exhibit a much lower phenocopy rate). For genetically directed RNAi mediated by expression of inverted repeat genes, we selected 50 transgenic roller L4s from lines harboring various hsp-16ₚ(IR) constructs plus co-transformation plasmid pRF423 (array transmission frequency >60%) and reared continuously at 20° C. (non-heat shock) or heat shocked for 4 hours at 35° C., before returning to 20° C. Progeny of these animals were scored for phenotypes of interest at embryonic or larval stages as appropriate; behavioral assays and phenotypic analysis as indicated in Table 3 and Figure legends. On average at least half of lines for a given gene assayed conferred potent interference upon heat-activation.

The following examples are provided to illustrate various embodiments of the invention. They are not intended to limit the invention in any way.

### EXAMPLE I

To test the feasibility of specific gene disruption via in vivo expression of double stranded RNA, we constructed transgenic nematodes that synthesize hairpin ds RNA³ from IR genes under the control of the strong heat shock-inducible promoter, hsp16-2 ⁶⁻⁸ (FIG. 2). We first compared effects of conventional RNAi via injection of dsRNA, expression of sense and antisense genes, and in vivo production of dsRNA using C. elegans predicted gene C37A2.5, an essential gene required for progression past the L2 larval stage (N. Tavernarakis, S. Wang, M., Driscoll, unpublished observations). Conventional RNAi via injection of C37A2.5 dsRNA¹ produces a high yield of L2 stage-arrested F1 progeny (Table 3). Expression of the antisense strand, which can be an effective method for specific gene inactivation⁹, confers a modest percentage of phenocopy progeny, whereas expression of the sense stand is ineffective. To test in vivo RNAi, we heat-shocked young adults of transgenic lines harboring extrachromosomal hsp16-2ₚC37A2.5(IR). In vivo promoter-driven RNAi proved effective in reproducing the C37A2.5 null phenotype, with efficiencies approaching that of direct injection of dsRNA (Table 3). Likewise, promoter-driven RNAi efficiently disrupted the Mi-2 chromatin remodeling homolog F2612.7¹⁰ to phenocopy the sterile phenotype of a deletion of this gene (Table 1).

We conclude that in vivo driven RNAi can be effective and that this technique should enable convenient generation of large populations of phenocopy mutants, even when development or reproduction is blocked.

TABLE 3
In vivo dsRNA interference

| Gene disruption approach | Trial/Line 1 | Trial/Line 2 | Trial/Line 3 | Trial/Line 4 |
|---|---|---|---|---|
| dsRNA C37A2.5 | 94 ± 8 | 89 ± 4 | 97 ± 5 | 89 ± 7 |
| injected | | | | |
| pPD49.78 (hsp16-2ₚ alone) + heat | 0 | 0 | 0 | 0 |
| shock | | | | |
| hsp16-2ₚC37A2.5 sense + heat | 0 | 0 | 0 | 0 |
| shock | | | | |
| hsp16-2ₚC37A2.5 antisense + heat | 9 ± 4 | 9 ± 4 | 11 ± 6 | — |
| shock | | | | |
| hsp16-2ₚC37A2.5(IR) − heat | 0 | 0 | 0 | 0 |
| shock | | | | |
| hsp16-2ₚC37A2.5(IR) + heat | 67 ± 3 | 79 ± 6 | 84 ± 5 | 56 ± 7 |
| shock | | | | |
| hsp16-2ₚF26F12.7(IR) − heat | 1 ± 0.9 | 2 ± 1 | 1 ± 0.9 | 3 ± 1.3 |
| shock | | | | |
| hsp16-2ₚ F26F12.7(IR) + heat | 58 ± 4 | 59 ± 5 | 75 ± 8 | 82 ± 6 |
| shock | | | | |
| ds mec-4 RNA | 12 ± 7 | 19 ± 5 | 15 ± 6 | — |
| injected | | | | |
| hsp16-2ₚmec-4(IR) − heat | 0 | 0 | 0 | — |
| shock | | | | |
| hsp16-2ₚmec-4(IR) + heat | 58 ± 4 | 60 ± 7 | 61 ± 8 | — |
| shock | | | | |
| ds unc-8 RNA | 0 | 0.8 ± .01 | 0 | 0 |
| injected | | | | |
| hsp16-2ₚunc-8(IR) − heat | 0 | 0 | 0 | 0 |
| shock | | | | |
| hsp16-2ₚunc-8(IR) + heat | 17 ± 3 | 11 ± 5 | 14 ± 2 | 13 ± 3 |
| shock | | | | |
| Results for four injection trials using conventional RNAi or heat shock induced in vivo RNAi in 4 transgenic lines (unless otherwise noted) are indicated. In all experiments, at least 100 animals were scored per experimental trial. Gene C37A2.5 is required for developmental progression past the L2 stage. Numbers indicate the percentage of F1 progeny arrested at the L2 stage ± SD. Co-expression of sense and antisense genes, which can be effective²⁴, | | | | |
| # was not tested. Deletion of chromatin remodeling gene homolog F26F12.7 causes sterility (S. Wang and M. Driscoll, unpublished). Treated progeny of transgenic lines harboring hsp16-2ₚF26F12.7(IR) were scored for % that fail to develop into fertile adults. A similar strategy for in vivo disruption of a second MI-2 homolog, T14G8.1, yielded 59% and 72% sterile in progeny of two lines scored after heat shock (data not shown). mec-4 is expressed in six | | | | |
| # mechanosensory neurons and is required for touch sensitivity. ds mec-4 RNA or plasmid hsp16-2ₚmec-4(IR) was introduced into wild type animals and progeny were scored for touch insensitivity. unc-8(n491) is a dominant gain-of-function mutation that causes coiling and backward paralysis; locomotion in a loss of function mutant is nearly normal¹⁵. ds unc-8 RNA or plasmid hsp16-2ₚunc-8(IR) was introduced into the n491 background and progeny were assayed for | | | | |
| # backing proficiency. Note that to regain backing ability, gene expression must be knocked down in the majority of unc-8-expressing cells, approximately 60 neurons. | | | | |

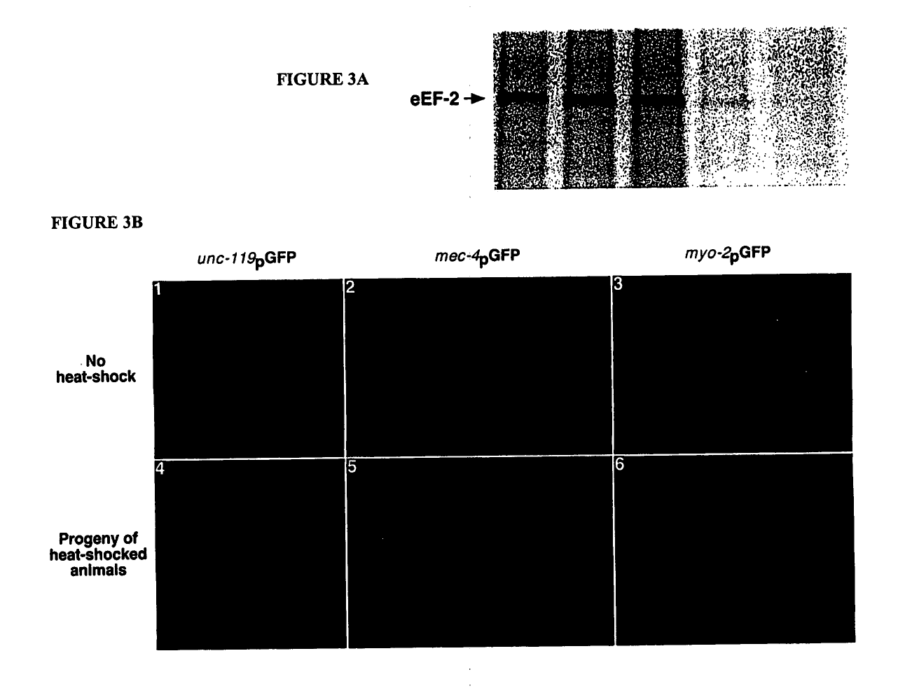

C. elegans translation elongation factor 2 kinase eEF-2 (efk-1)¹¹ phosphorylates eEF-2, an activity abolished by a Tc1 insertion into the active site (A. Ryazanov, C. Mendola, L. Zhang and J. Culotti, unpublished observations) (FIG. 3a). We find that kinase activity in the offspring of heat-shocked hsp16-2ₚefk-1 (IR) transgenic parents is reduced at least several fold in 4/6 lines we assayed. An analogous assay could not be performed on a population of phenocopy mutants induced by conventional RNAi, since several hundred animals are required. We conclude that inducible IR genes are effective in generating populations amenable to biochemical analysis.

Injected dsRNA is not uniformly effective in disrupting gene expression in the nervous system. For example, we find that only 6/210 progeny from three lines harboring integrated unc-119ₚGFP (expressed in all neurons) injected with double-stranded GFP RNA exhibited a detectable reduction in fluorescence (FIG. 3b). To examine more closely effects of endogenously expressed dsRNA species on gene inactivation in the differentiated nervous system, we first constructed a plasmid that directs in vivo expression of double-stranded GFP RNA upon heat shock and tested for extinction of fluorescent signals generated by cell-specific GFP reporter fusions (FIG. 3b). We co-introduced the hsp16-2ₚGFP(IR) construct and unc-119ₚGFP (pIM175¹²; expressed at high levels throughout the nervous system¹³), selected lines exhibiting strong GFP fluorescence, heat shocked in the L4 stage, and examined fluorescence in their progeny. Approximately 79% of roller progeny from 3 (of 5) lines harboring unc-119ₚGFP and hsp16-2ₚGFP(IR) exhibit easily distinguished knockdown effects, with fewer than 10 cells readily detectable in most (FIG. 3b). We did not detect any consistent pattern of cells that appeared refractory to fluorescence inactivation, suggesting that all cells in the nervous system are susceptible to the effects of in vivo RNAi.

We also tested effects of heat shock induction of hsp16-2ₚGFP (IR) on expression of an integrated mec-4ₚGFP gene, which is specifically expressed in the six touch receptor neurons¹⁴. On average, 85% of roller progeny of heat shocked parents harboring the extragenic hsp-16ₚGFP(IR) transgene had GFP signals that were either eliminated or markedly attenuated (2 of 4 lines; FIG. 3b). By contrast, we observed similar effects in only 11 of 270 progeny of a line harboring an integrated mec-4ₚGFP reporter injected with dsGFP RNA.

We also tested for dsRNA-mediated inactivation of C. elegans neuronal genes. Conventional RNAi mediated by introduced mec-4 dsRNA induced touch-insensitivity in 46/300 (15%) offspring of injected wild type parents. On average, 60% progeny of heat-shocked lines harboring hsp16-2ₚmec-4 (IR) were touch insensitive (Table 1). As another example, we tested the effectiveness of in vivo-directed RNAi in the inactivation of unc-8, a neuronally expressed gene that in our hands has been resistant to the effects of conventional RNAi. unc-8 gain of function allele n491 dominantly induces uncoordinated locomotion characterized by the inability to back up; the loss of function phenotype appears nearly wild type¹⁵. Injection of unc-8 dsRNA is not effective in knocking out the gf phenotype (2 phenocopy mutants generated among 1300 progeny of injected parents). Progeny of heat shocked unc-8(n491) mutants harboring hsp16-2ₚunc-8(IR) are effectively targeted about 13% of the time (Table 3). Taken together, our results indicate that sequences expressed in terminally differentiated neurons can be targeted by in vivo induced RNAi and in some instances effects are more potent than those observed after injection of dsRNA. For all nine cases we investigated, heat shock of control lines carrying the expression vector alone or low temperature growth of lines carrying the hsp16-2ₚ(IR) genes did not produce any readily apparent phenotypes (we assayed for the anticipated knockout phenotype, morphological and locomotion defects, fertility, and developmental abnormalities; >100 animals examined per line). Thus, effects of in vivo RNAi appear highly specific, consistent with reported tight regulation of the hsp16-2 promoter⁸ and the selective precision of RNAi knock out capacity¹. Moreover, in vivo RNAi appears effective in many tissue types, including neurons (FIG. 2b, note that C37A2.5 and efk-1 are expressed early in development and later in a broad range of cells including body wall and pharyngeal muscles, neurons, hypodermis and intestine (N. Tavernarakis, A. Ryazanov and M. Driscoll, unpublished); Mi2 homolog F2612.7 is expressed in the hypodermis; Mi2 homolog T14G8.1 is expressed in the hypodermis and pharynx (S. Wang, N. Tavernarakis and M. Driscoll unpublished); myo-2 is expressed in pharyngeal tissue¹⁶).

Our analysis establishes that endogenous inverted repeat genes can be expressed to generate dsRNA species with biological effects similar to, and superior than that of directly injected dsRNA. Advantages of expressing heritable inverted repeat genes include that: 1) stable lines harboring the potential for gene inactivation can be easily maintained, 2) assays requiring large numbers of mutant phenocopies are feasible, and 3) inhibition can be inducible, and thus may be used for stage-specific gene inactivation. In some cases, the endogenous high level of dsRNA product produced upon heat shock appears to make for more potent inhibition than germline injected dsRNA. Although we have focused our initial studies on the use of the inducible hsp16-2 promoter, our findings indicate that it us possible to inactivate specific genes for the duration of their expression period by integrating a transgene in which the promoter of the gene of interest drives transcription of an inverted repeat segment of the same gene. In addition, since dsRNA can inactivate genes in flies, plants, trypanosomes and planaria¹⁷⁻²⁰, in vivo directed RNAi could be effective in other organisms. These observations indicate that a similar strategy for in vivo driven RNAi can be applied to inactivate specific genes in organisms that can be genetically engineered but are not readily amenable to direct injection of dsRNA.

### EXAMPLE II

### IR Constructs for Use in the Prevention and Treatment of Neurodegenerative Disorders

Neurodegenerative disorders, such as Alzheimer's and Parkinson's disease disproportionately affect the elderly, the most rapidly growing sector of the population. Additionally, many neural viral infections, such as those caused by HIV and encephalitis viruses, cause irreversible destruction of brain tissue, thereby compromising the quality of life for the patient. Accordingly prophylactic and therapeutic treatments are highly desirable for preventing and/or inhibiting the neurodegeneration associated with such diseases.

Neurodegenerative disorders are generally classified as heritable or spontaneous in origin. Parkinson [symbol] s disease (PD) and Alzheimer's disease (AD), appear to be both heritable and spontaneous diseases. Disorders such as spinocerebellar ataxia 1 and 3 are heritable diseases. Many of these disorders are characterized by proteinaceous cellular inclusions (either inside or outside cells) encoded by genes whose expression is implicated in disease. In all cases, inappropriate accumulation of such proteinaceous cellular inclusions is associated with the progression of the disease and, in some cases, is causally linked to disease etiology.

During viral infection and replication in brain tissue, harmful viral proteins accumulate and cellular destruction occurs. In HIV infection, CD4+ T cells are targeted for destruction. During viral encephalitis, neuronal cells are destroyed. Inhibition of viral replication can effectively prevent this type of neuronal cell damage.

Given that the aberrant, toxic accumulation of a cellular protein(s) to toxic levels is a theme common to diseases mentioned above and many other disorders, therapeutic treatments designed to reduce levels of such toxic proteins have general utility in the treatment of patients suffering from neurodegenerative disorders. The present example provides compositions and methods suitable for reducing the levels of such toxic proteins utilizing RNAi generated from inheritable inverted repeat (IR) gene constructs. Such compositions can be employed as a single agent or can be utilized in combination with other therapies. Such therapeutic approaches should result in an amelioration of symptoms associated with the disease and potentially reverse the course of the disease by eliminating the causative agent. Moreover, the compositions and methods of the present invention may be used to advantage prophylactically to delay or prevent the onset of a disorder. This application has particular utility for preventing or delaying the onset of disease in patients with a known genetic predisposition for a specific heritable neurodegenerative disorder.

In the present example, the usefulness of the compositions and methods of the invention for the treatment of Alzheimer's disease (AD) and Parkinson's disease is demonstrated.

AD is a spontaneous neurodegenerative disorder which is caused by cell death in the brain and is characterized by the deposition of amyloid plaques. A major component of these plaques is β-amyloid, which is a cleavage product of the amyloid precursor protein (APP). Brain cell death is associated with an increase in a 42 amino acid fragment of β-amyloid, called Aβ, which is generated by an aberrant processing event of APP. A critical goal in AD therapy is the development of therapeutic agents which effectively reduce the production of this fragment.

To achieve this goal, an IR construct containing a fragment of DNA encoding Aβ can be placed under the control of a brain specific promoter. Following administration to a patient, which can be achieved by specific delivery to the brain or systemic introduction (see above), expression of the Aβ inverted repeat double stranded RNA molecule should dramatically reduce production of this neurotoxic Aβ fragment. This, in turn, should result in a dramatic reduction in plaque formation and delay or prevent disease onset.

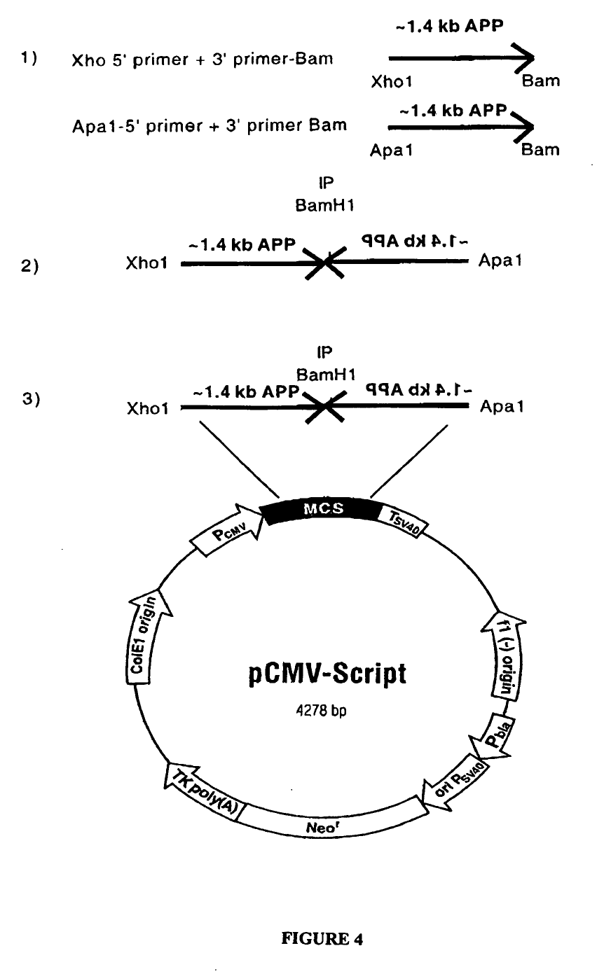

An AS-IR expression construct can be generated as follows. A 1 kb fragment of Aβ nucleic acid is obtained. The GenBank Accession number for the genomic sequence of Aβ is D87675. The Genbank accession number for the cDNA is Y00264. The sequence can be amplified using Aβ specific primers that incorporate unique restriction sites at the IR fragment 5′ and 3′ ends and another restriction site to generate the inverted repeat which is ultimately situated at the inversion point (IP). See FIG. 4. The 5′ and 3′ terminal restriction sites (designated as end A and B) can be used to insert the Aβ inverted repeat into an expression vector. Two Aβ products can then be generated in parallel by PCR amplification of human cDNA using (1)primers A and IP and (2) primers B and IP, respectively. Amplified Aβ fragments A-IP and B-IP can be digested with the appropriate restriction enzyme located at the IP restriction site (IPRS) to generate compatible termini which could then be ligated to produce an Aβ-IR. Digestion of the Aβ-IR at the 5′ and 3′ restriction sites A and B facilitates ligation into an expression vector that has been linearized with restriction enzymes to generate compatible sites. The New England Biolabs catalog provides a wide variety of restriction enzymes and the corresponding restriction sites which can be utilized in the construction of the IR repeat constructs of the invention.

In an alternative cloning strategy, two naturally occurring restriction sites found within DNA encoding the Aβ fragment could be used to excise an Aβ fragment. In a two-step ligation reaction, this Aβ DNA fragment could be ligated end-to-end to generate an inverted repeat, and consequently ligated into an appropriately linearized vector as described above. In this cloning strategy it is necessary to treat the linearized expression vector with calf intestine alkaline phosphatase.

In yet another approach each of the desired sequence elements for the IR construct may be synthesized separately, blunt ended and then ligated using DNA ligase.

A variety of vectors are available for expressing exogenous nucleic acids in human cells. The plasmid vector pCEP4 (Invitrogen), for example, is comprised of components that facilitate selection and expression in human cells. pCEP4 contains the following elements: a CMV promoter, a TKpA—thymidine kinase polyadenylation signal, a Hygromycin resistance gene, a ColE1 origin, an Ampicillin resistance gene, an Epstein Barr Virus Nuclear Antigen (EBNA-1) and an EBV origin (OriP EBV) for episomal replication in EBV transformed cell. In another example, the plasmid clone pCR3 (Invitrogen) could also be used to drive high level IR gene expression in mammalian cells. This plasmid vector includes a CMV promoter—Cytomegalovirus immediate—early promoter for high-level expression of the cloned IR gene; BGHpA—Bovine growth hormone polyadenylation signal for mRNA stability; ColE1—origin for replication, maintenance, and high copy number in E. coli; TKPA—thymidine kinase polyadenylation signal; Neomycin—neomycin resistance gene for selection of stable mammalian cell lines; PSV40/ori—origin for episomal replication in cells containing the SV40 large T antigen; Ampicillin—resistance gene for selection and maintenance in E. coli; F1 ori-origin for rescue of sense strand for mutagenesis and single strand sequencing.

In another example, the CMV-Script-Ex vector (Stratagene) could also be used to drive high level IR gene expression in mammalian cells. Several vectors suitable for use in the present invention are set forth in Table II.

Using Alzheimer's disease as an exemplary disease model, and the β-amyloid encoding nucleic acid as the target for inhibition, the selected nucleic acid sequence corresponds to approximately 1000 contiguous nucleotides from the sequences set forth in the Genbank Accession Nos. provided above, operably linked in a sense and antisense orientation. An exemplary expression construct for use in inhibiting the expression of beta-amyloid protein is shown in FIG. 4.

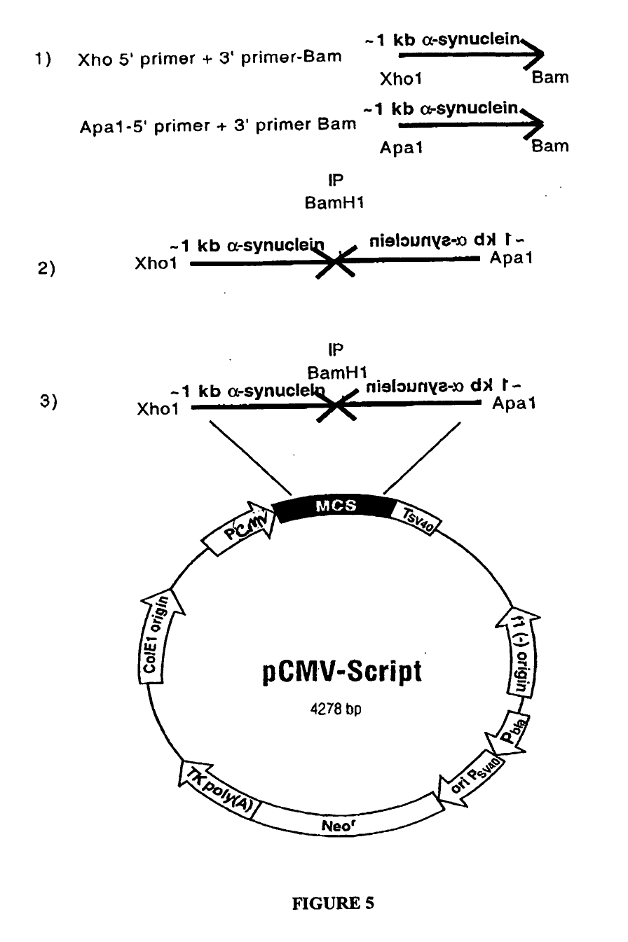

As mentioned previously, the compositions and methods of the invention also have utility in the treatment and prevention of Parkinson's disease. For example, alpha-synuclein has been implicated in the pathology of familial Parkinson's disease. The nucleic acid sequence encoding the alpha-synuclein protein is known. See Genbank Accession No. D31839. Accordingly, an IR construct can be generated in accordance with the present invention to reduce alpha synuclein accumulation in the affected patient. An appropriate expression vector for this purpose is shown in FIG. 5, which contains 1081 nucleotides from GenBank Accession No. D31839 operably linked in a sense and antisense orientation. Methods for delivering the alpha-synuclein targeted IR constructs of the invention to the dopaminergic neurons of the substania nigra (the neurons affected in the disease) are set forth hereinabove.

### EXAMPLE III

### Use of IR Gene Construct Expression Vectors for Controlling Plant Disease

Geminiviruses are plant pathogens that infect a wide range of vegetable crops in tropical and subtropical regions with devastating consequences (Brown et al. (1992) Plant Disease, 76:220-225). Major epidemics of geminivirus infections of beans and tomatoes have occurred recently on several continents, thereby threatening the livelihood of farmers producing these crops and causing shortages which adversely impact the consumer population. The intransigent nature of the problem is underscored by a failure to generate cultivars that are resistant to geminiviruses by traditional breeding methods.

The compositions and methods of the invention are suitable for generating geminivirus resistant strains of a variety of vegetable crops that are susceptible to this family of viruses. In brief, the heritable system of RNAi described herein facilitates the generation of transgenic plant strains that produce dsRNA molecules which inhibit the expression of viral genes critical for productive infection. Transgenic plant strains can be engineered to express the inhibitory RNA molecule under the control of either a constitutive or an inducible promoter. In one embodiment of the invention, an IR construct containing a fragment of DNA encoding an essential geminivirus protein may be placed under the control of a constitutive promoter that functions in plant cells. Viral genes encoding suitable target proteins for such inhibition include, but are not limited to, those essential for viral replication and capsid assembly. Plant cells transformed with such an IR gene construct expression vector should be resistant to viral infection. Progeny of plant cells containing stably integrated IR gene construct expression vectors inherit resistance to geminivirus infection. In a particularly preferred embodiment of the invention, the transformed plant cell is a seed from which resistant plant stock can be derived.

In the present example, an IR gene construct expression vector is described which can confer disease resistance to the tomato yellow leaf curl (TYLC) geminivirus. The TYLC virus infects the cultivated tomato (Lycopersicon esculentum), with devastating consequences. The virus which has a single monopartite genome, is a subgroup III type of geminivirus, transmitted by the whitefly. Navot, N. et al., (1991) Virology, 151-161. Notably, a whitefly-transmitted TYLC-like geminivirus having a bipartite genome has also been cloned. See Rochester, D. E., et al., (1990) Virology, 520-526.

The TYLC viral genome comprises six open reading frames. Two open reading frames, V1 and V2, are located on the virion or plus strand, whereas the four remaining open reading frames, C1, C2, C3, and C4 are located on the complementary or minus strand. The C2 open reading frame displays partial overlap with the C1 and C3 open reading frames. The C1 open reading frame, which is sometimes referred to as AC1, includes the C4 open reading frame.

Partial or complete sequence data are available for several TYLC viral isolates. The entire genome of an Israeli isolate of TYLC virus, for example, has been cloned and sequenced. Navot, N. et al., (1991) Virology, 151-161. Sequence data are also available for TYLC viral isolates from Sardinia, Australia, Thailand, Egypt, and Sicily. Kheyr-Pour, A., et al., (1991) Nucl. Acids Res., 19:6763-6769. Dry et al. (1993) J Gen. Virol.74:147-151. Padidam et al. (1995) J. Gen. Virol. 76:249-263. The entire genome sequence of the Tomato yellow leaf curl virus can be found at GenBank Accession No. NC_{—}001996.

In an exemplary IR gene construct expression vector, the geminivirus Nia-protease (NP) gene, which is required for productive infection is targeted for gene silencing. A fragment of DNA which encodes a portion of the NP can be generated from either PCR of available TYLC isolates or by RT-PCR of RNA derived from infected plant cells amplified using suitable forward and reverse primers. Suitable primer sequences corresponding to NP (of approximately 20-25 bases) can be selected from sequence information available in a variety of data bases. The NP specific primers are designed to incorporate unique restriction sites at the IR fragment 5′ and 3′ ends and another unique restriction site to generate the inverted repeat which is ultimately situated at the inversion point (IP) (See figure). The 5′ and 3′ terminal restriction sites (designated as end A and B) can be used to insert the NP inverted repeat into a plant expression vector of choice. Specifically, two NP products would be generated in parallel by PCR amplification of TYLC genomic DNA or cDNA using (1) primers A and IP and (2) primers B and IP, respectively. Amplified NP fragments A-IP and B-IP would be digested with the appropriate restriction enzyme located at the IP restriction site (IPRS) to generate compatible termini which could then be ligated to produce an NP-IR. Digestion of the NP-IR at the 5′ and 3′ restriction sites A and B facilitates ligation into a plant expression vector that has been linearized with restriction enzymes to generate compatible sites.

In an alternative cloning strategy, two naturally occurring restriction sites found within an NP gene fragment could be used to excise the fragment. In a two-step ligation reaction, this NP fragment could be ligated end-to-end to generate an inverted repeat, and consequently ligated into an appropriately linearized expression vector as described above. In this cloning strategy it is suggested to treat the linearized expression vector with calf intestine alkaline phosphatase to prevent the recircularization of vector without the incorporation of the desired NP-IR insert.

In yet another approach, each of the IR gene construct expression vector sequence elements may be cloned and isolated separately and then operably linked into an expression vector using DNA ligase. As mentioned previously, the first and second coding regions of the IR gene construct may optionally include a spacer sequence between the first and second coding sequences to facilitate expression or stability of the clone.

A variety of plant expression vectors are available that would be of utility for the present invention. In a preferred embodiment of the invention, the cauliflower mosaic virus derived expression vector CMV35S could be utilized to drive expression of the NP-IR in tomato cells (Gleave, 1992).

Once fully constructed, the NP-IR expression vector would then be transformed into a suitable bacterial strain such as, for example, E. coli strain DH5 or SURE. Transformation into bacteria facilitates the generation of large quantities NP-IR expression vector, which can be used in the production of TYLC resistant tomato plant strains.

In one application of the present invention, tomato plants are transformed using Agrobacterium which requires a T-DNA producing binary vector (pBIN19 for example, Stanford et al., 1990). In another application of the present invention, tomato plants can be transformed using particle bombardment, for which there are no requirements for specific DNA constructs.

Methods are available for the isolation of stable plant transformants. In a preferred embodiment, the IR gene construct expression vector optionally includes a selectable marker gene. Such markers include, but are not limited to, the NPTII gene which confers resistance to the antibiotic kanamycin or the BAR gene which confers resistance to the plant herbicide Bialophos. Examples of tobacco vectors are: pART7 and pART 27 (Gleave, 1992 Plant Mol. Biol. 20: 1203-1207). A suitable rice vector is pVec4 (Wang et al., 1998 Acta Hortic. 461:401-405.).

### References

1. Fire, A. et al. Potent and specific genetic interference by double-stranded RNA in Caenorhabditis elegans. Nature 391, 806-811 (1998).

2. Montgomery, M. K., Xu, S., & Fire, A. RNA as a target of double-stranded RNA-mediated genetic interference in Caenorhabditis elegans. Proc. Natl. Acad. Sci. USA 95, 15502-15507 (1998).

3. Timmons, L. & Fire, A. Specific interference by ingested dsRNA. Nature 395, 854 (1998).

4. Montgomery, M. K. & Fire, A. Double-stranded RNA as a mediator in sequence-specific genetic silencing and co-suppression. Trends Genet. 14, 255-258 (1998).

5. Tabara, H., Grishok, A. & Mello, C. C. RNAi in C. elegans—Soaking in the genome sequence. Science 282, 430-431 (1998).

6. Jones, D., Russnak, R. H., Kay, R. J. & Candido, E. P. M. Structure, expression and evolution of a heat-shock gene locus in C. elegans that is flanked by repetitive elements. J. Biol. Chem. 261, 12006-12015 (1986).

7. Stringham, E. G., Dixon, D. K., Jones, D. & Candido, E. P. M. Temporal and spatial expression patterns of the small heat shock (hsp-16) genes in transgenic Caenorhabditis elegans. Mol. Biol. Cell 3, 221-233 (1992).

8. Fire, A., Harrison, S. W., & Dixon, D. A modular set of lacZ fusion vectors for studying gene expression in Caenorhabditis elegans. Gene 93, 189-198 (1990).

9. Lu, X. & Horvitz, H. R. lin-35 and lin-53, two genes that antagonize a C. elegans Ras pathway, encode proteins similar to Rb and its binding protein RbAp48. Cell 95, 981-991 (1998).

10. Zhang. Y., LeRoy, G., Seelig, H. P., Lane, W. S. & Reinberg, D. The dermatomyositis-specific autoantigen Mi2 is a component of a complex containing histone deacetylase and nucleosome remodeling activities. Cell 95, 279-89 (1998).

11. Ryazanov, A. G. et al. Identification of a new class of protein kinases represented by eukaryotic elongation factor-2 kinase. Proc. Natl. Acad. Sci. USA 94, 4884-4889 (1997).

12. Maduro, M. & Pilgrim, D. Identification and cloning of unc-119, a gene expressed in the Caenorhabditis elegans nervous system. Genetics 141, 977-988 (1995).

13. Ren, X. -C., Kim, S., Fox, E., Hedgecock, E. & Wadsworth, W. G. Role of netrin UNC-6 in patterning the longitudinal nerves of Caenorhabditis elegans. J. Neurobiol. 39, 107-118 (1999).

14. Mitani S., Du, H., Hall, D., Driscoll, M. & Chalfie, M. Combinatorial control of touch receptor neuron expression in Caenorhabditis elegans. Development 119, 773-783 (1993).

15. Park, E. -C. & Horvitz, H. R. Mutations with dominant effects on the behavior and morphology of the nematode C. elegans. Genetics 113, 821-852 (1986).

16. Dibb, N. J., Maruyama, I. N., Krause, M. & Karn, J. Sequence analysis of the complete Caenorhabditis elegans myosin heavy chain gene family. J. Mol. Biol. 205, 603-613 (1989).

17. Kennerdell, J. R. & Carthew, R. W. Use of dsRNA-mediated genetic interference to demonstrate that frizzled and frizzled 2 act in the wingless pathway. Cell 95,1017-1026 (1998).

18. Ngo, H., Tschudi, C., Gull, K. & Ullu, E. Double-stranded RNA induces mRNA degradation in Trypanosoma brucei. Proc. Natl. Acad. Sci. USA 95,14687-14692 (1998).

19. Voinnet, O., Vain, P., Angell, V. & Baulcombe, D. C. Systemic spread of sequence-specific transgene RNA degradation in plants is initiated by localized introduction of ectopic promoterless DNA. Cell 95, 177-187 (1998).

20. Sanchez Alvarado. A. & Newmark, P. A. Double-stranded RNA specifically disrupts gene expression during planarian regeneration. Proc. Natl. Acad. Sci. USA 96, 5049-5054 (1999).

21. Brenner, S. The genetics of Caenorhabditis elegans. Genetics 77, 71-94 (1974).

22. Mello, C. C. & Fire, A. DNA transformation. in, Methods in Cell Biology. Caenorhabditis elegans: Modern Biological Analysis of an Organism. H. F. Epstein & D. C. Shakes (Eds). (Academic Press, Inc. San Diego, 1995) pp. 451-482.

23. Kramer, J. M., French, R. P., Park, E. -C. & Johnson, J. J. The Caenorhabditis elegans rol-6 gene, which interacts with the sqt-1 collagen gene to determine organismal morphology, encodes a collagen. Mol. Cell. Biol. 10, 2081-2089 (1990).

24. Tabara, H. et al. The rde-1 gene, RNA interference, and transposon silencing in C. elegans. Cell 99, 123-132 (1999).

While certain preferred embodiments of the present invention have been described and specifically exemplified above, it is not intended that the invention be limited to such embodiments. Various modifications may be made to the invention without departing from the scope and spirit thereof as set forth in the following claims.

1. An IR gene construct encoding an inverted repeat gene, said construct comprising:
a) a promoter element operably linked in a 5′ to 3′ direction to a first coding sequence and a second sequence, said first coding sequence being in a sense orientation, said second sequence being the first coding sequence in an antisense orientation; and
b) a transcription termination element operably linked 3′ to said first and second coding sequences.

2. The IR gene construct of claim 1 inserted into an expression vector.

3. A IR gene construct as claimed in claim 1, said promoter being inducible.

4. A IR gene construct as claimed in claim 2, said promoter being selected from the group consisting of a heat shock promoter, a metallotheinine promoter, a glucocorticoid promoter, CMV promoter, SV40 promoter, nervous system specific promoters, unc-119, mec-4, odr-4, muscle promoters unc-54, myo-2, act-l and ben-1, and a CaMV promoter.

5. The DNA construct of claim 1, wherein a spacer sequence is inserted between said first coding and second sequences.

6. A host cell containing the DNA construct of claim 1.

7. A method for production of a phenocopy knock out mutant by introducing an inverted repeat gene into an organism, said inverted repeat gene comprising:
a) a promoter element operably linked in a 5′ to 3′ direction to a first coding sequence and a second sequence, said first coding sequence being in a sense orientation, said second sequence being the first coding sequence in an antisense orientation; and
b) a transcription termination element operably linked 3′ to said first and second coding sequences.

8. A method as claimed in claim 7, wherein said inverted repeat gene is introduced into C. elegans via a process selected from the group consisting of microinjection, soaking, and DNA coated particle bombardment.

9. A method as claimed in claim 7, wherein said inverted repeat gene contains the coding sequence of a gene selected from the group consisting of green fluorescent protein gene, C3782.5, F26F12.7, T14G8.1, efk-1, mec-4, unc-8, unc-119, degenerinis ZB770.1, T28B8.5, T28F24.2, C.24G7.2 and T28D9.7.

10. A method as claimed in claim 7, wherein said inverted repeat gene is passed onto progeny thereby generating phenocopy mutants upon induction of expression of said inverted repeat gene.

11. A method as claimed in claim 7, wherein said organism is selected from the group consisting of plants, mice, humans, insects and nematodes.

12. An IR gene construct expression vector as claimed in claim 2 for the treatment of Alzheimers disease as shown in FIG. 4.

13. An IR gene construct expression vector as claimed in claim 2 for the treatment of Parkinson's disease as shown in FIG. 5.

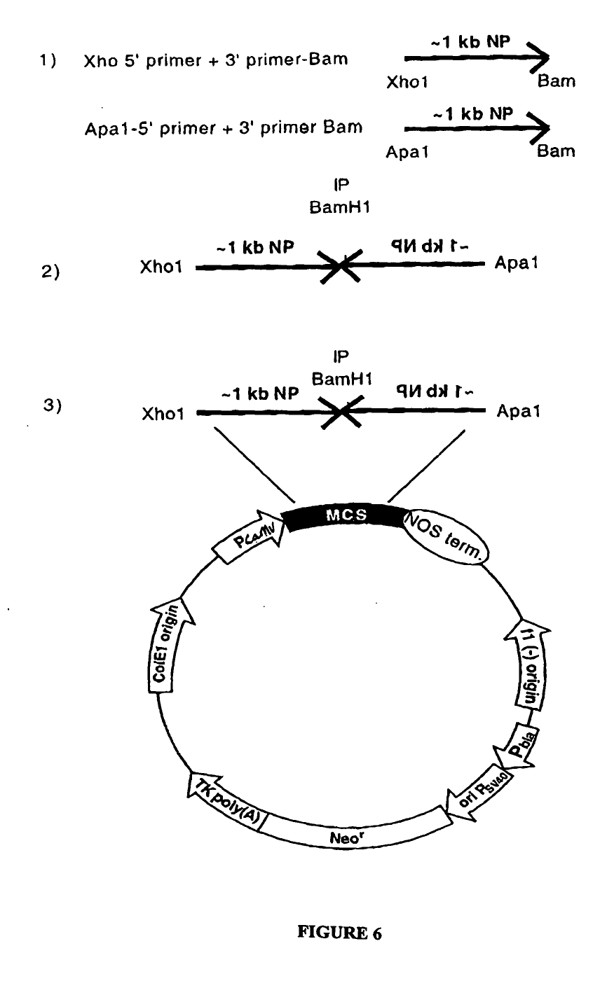

14. An IR gene construct expression vector as claimed in claim 2 for the treatment of tomato leaf curl geminivirus as shown in FIG. 6.
